(19) United States
(12) Patent Application Publication
(10) Pub. No.: US 2007/0240984 A1
(43) Pub. Date: Oct. 18, 2007

(54) Biosensors comprising heat sealable spacer materials

(76) Inventors: Natasha D. Popovich, Pompano Beach, FL (US); Dennis Slomski, Wellington, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/405,537

(22) Filed: Apr. 18, 2006

(51) Int. Cl. G01N 33/487

(52) U.S. Cl. 204/403.01

(57) ABSTRACT

Disclosed herein is a biosensor for measuring analyte in a fluid that comprises a substrate layer having disposed thereon at least one each of an electrode, cathode, anode, and a novel spacer material. The spacer material according to the present disclosure comprises a heat sealable organic layer that covers at least a portion of the anode and defines at least one edge of the anode, wherein the spacer material has at least one hole punched through it and defines a cavity or well for accepting chemistry. Also disclosed is a method of making such biosensors.

The present disclosure relates to biosensors for measuring an analyte in a bodily fluid, such as blood, wherein the biosensor comprises a heat sealable, organic spacer material that particularly defines at least one edge of a working electrode disposed on the biosensor. The present disclosure also relates to methods of making the biosensor and methods of measuring analytes in bodily fluid using the biosensor.

Electrochemical sensors have long been used to detect and/or measure the presence of analytes in a fluid sample. In the most basic sense, electrochemical sensors comprise a reagent mixture containing at least an electron transfer agent (also referred to as an “electron mediator”) and an analyte specific bio-catalytic protein, and one or more electrodes. Such sensors rely on electron transfer between the electron mediator and the electrode surfaces and function by measuring electrochemical redox reactions. When used in an electrochemical biosensor system or device, the electron transfer reactions are transformed into an electrical signal that correlates to the concentration of the analyte being measured in the fluid sample.

Electrochemical glucose sensors are based on measurement of current resulting from oxidation of a reduced form of the mediator, generated by reactions between the glucose molecule, an oxidoreductase and the oxidized form of the mediator. Signal measured at a glucose sensor is directly proportional to the anode area; hence, precision of a blood glucose test/device can be directly correlated to the anode area definition and control. If the edges of an electrode are irregular and vary from medium to medium, the area of the electrode, and therefore the measurement, will also vary from medium to medium. For these reasons, edges of the electrode are an important factor in developing more accurate biosensors with smooth edges being desirable to insure precision and accuracy of the measurement.

In addition to improved accuracy, spatial resolution of the electrode is important because the smaller the surface area of the electrode, the smaller the sample volume required. This is desirable with, for example, glucose monitoring for diabetics, where the patient must test his or her blood glucose multiple times a day. Smaller blood volume requirements allow the patient to obtain blood from areas with lower capillary densities than the fingers, such as the upper arm and forearm, which are less painful to lance.

One method currently used for manufacturing biosensors is screen printing. Screen printing involves laying a mesh screen with an electrode pattern onto a substrate and then spreading an electroactive paste over the screen. Because screen printing involves extruding the paste through the screen onto the substrate, it is difficult to obtain electrode patterns with small resolution and smooth edges. For example, in traditional screen printed glucose sensors anode area is defined by edges of the electrode carbon ink and dielectric ink. In addition, one additional layer is typically needed to form the sample well, and in many cases, this layer is also a screen printed dielectric ink. With current screen printing technology, a dielectric layer is needed to define the anode. Therefore, the area of the anode, and thus the accuracy of the resulting biosensor is a function of the method of depositing the dielectric layer, as well as the chemistry of this layer.

Coupled with the need to better define the anode area, is a desire to simplify manufacturing steps of the new generation of biosensors in order to provide a more robust process, high production yields and high quality sensors. New materials are being explored that could be beneficial in attaining this goal.

To solve the foregoing problems, the Inventors have developed a unique method of defining the anode area of a biosensor by utilizing a heat sealable spacer material to accurately define one or more edges of the anode instead of a dielectric layer. The Inventors have found that this method is particularly useful when used with a laser ablation technique. With the laser ablation technique, an electroactive material, such as gold is sputtered in a thin film onto a substrate. A laser then traces across the substrate and ablates the electroactive material, leaving an electrode pattern on the substrate. This technique produces electrodes with better resolution and smoother edges than with screen printing. In addition to greatly improving the accuracy and reproducibility of the anode area, the method of fabricating the biosensor is simpler than current process as it no longer requires depositing a separate dielectric layer.

### SUMMARY OF INVENTION

Disclosed herein are electrochemical biosensors for measuring analyte, such as glucose, cholesterol, lactate, acetoacetic acid (ketone bodies), theophylline, and hemoglobin A1c in a fluid. The inventive biosensors comprise a substrate layer comprising: at least one electrode; at least one cathode; at least one anode; and at least one spacer material. In one embodiment, the spacer material comprises a heat sealable organic layer that activates above 85° C. For example, the heat sealable organic film may comprise a polyester containing film, such as polyethylene terephthalate (PET) with a polyolefin layer disposed thereon.

Whatever the composition of the spacer material, it typically has at least one opening punched through it, and covers at least a portion of the working electrode, such as the anode. The punched opening defines at least one edge of the anode, and typically two opposing edges. The remaining two opposing edges are typically defined by ablated laser lines, and thus also have excellent edge quality.

In addition to defining edges of the anode, once it is applied to the substrate, the opening punched through the spacer material defines a cavity or well sufficient for accepting chemistry deposited on the assembled biosensors.

Also disclosed herein is a method of making the described biosensor. In one embodiment, the method comprises depositing an electroactive material onto a substrate to form a coated substrate. The electroactive material may comprise a conducting or semiconducting material. Patterns are next formed into the coated substrate layer by ablating the electroactive material with a laser. Such patterns form an electrode array comprising at least one electrode, cathode, and anode.

After the electrode array is formed, the spacer material is applied over the substrate, such that it covers at least a portion of array. As mentioned, the spacer material has a least one opening that is punched prior to being deposited on the substrate. The opening through the spacer material is positioned to ensure it covers at least a portion of the anode and defines at least one edge of the anode.

Once applied, the spacer film is laminated onto the substrate by applying heat and pressure at conditions sufficient to form a seal with the electrode array and substrate, thus forming an assembled biosensor. Next, the chemistry can be deposited within the cavity or well defined by the spacer material. Once the chemistry dries, a cover is applied over the sample cavities to form capillary gaps to which blood sample is drawn.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and, together with the description, serve to explain the principles of the invention.

### BRIEF DESCRIPTION OF THE DRAWINGS

FIG. 1 is an optical image of a biosensor (without cover) according to the present disclosure.

FIG. 2 is an SEM image of a punched spacer showing excellent edge definition and no adhesive extrusion.

FIG. 3 are optical CMM images of a punched spacer showing excellent (a) circular and (b) straight edge definition and no adhesive extrusion.

FIG. 4 are SEM images of a punched spacer showing excellent edge definition and no adhesive extrusion.

FIG. 5 is a histogram of a chronoamperometry test showing a coefficient of variation (% CV) of 0.85.

FIG. 6 are profilometry scans across the top of the punched spacer material laminated onto the electrode-containing substrate.

In accordance with the present disclosure provided herein are electrochemical biosensors developed for measuring an analyte in a non-homogenous fluid sample, such as a bodily fluid chosen from blood, urine, saliva and tears. The biosensor includes at least one or more electrodes and a reaction reagent system comprising an electron mediator and an oxidation-reduction enzyme specific for the analyte to be measured.

The biosensor may comprise a substrate layer that includes at least one electrode, at least one cathode, at least one anode, and at least one spacer material. In one embodiment, the biosensor comprises two fill detect electrodes, an anode and a cathode.

The spacer material typically comprises a heat sealable organic layer that covers at least a portion of the anode, such that it defines at least one edge of the anode. The heat sealable organic layer may further cover a portion of the electrode, or cathode, or a portion of both the electrode and cathode.

The heat sealable layer comprises a polymer that typically activates at or above 85° C. For example, the heat sealable organic layer may comprise a polyester containing film, such as polyethylene terephthalate (PET), with a polyolefin layer disposed thereon. The polyolefin layer may be disposed on the PET by a co-extrusion process or may be deposited via a spraying technique.

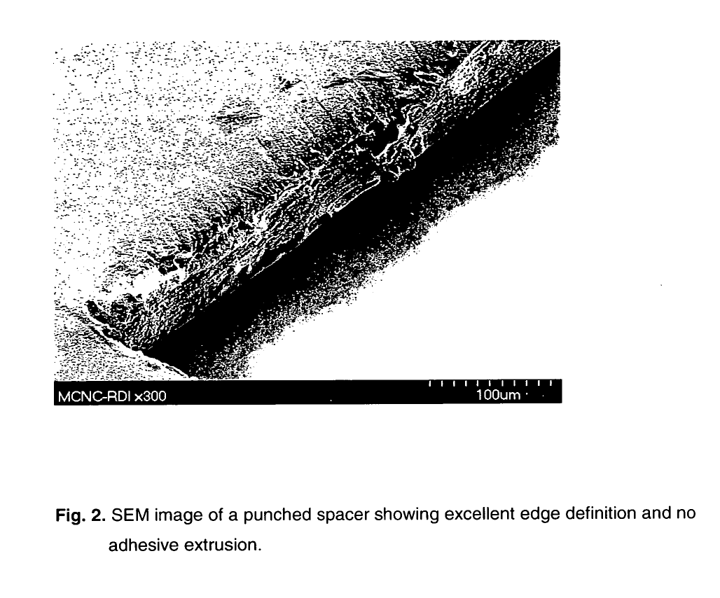

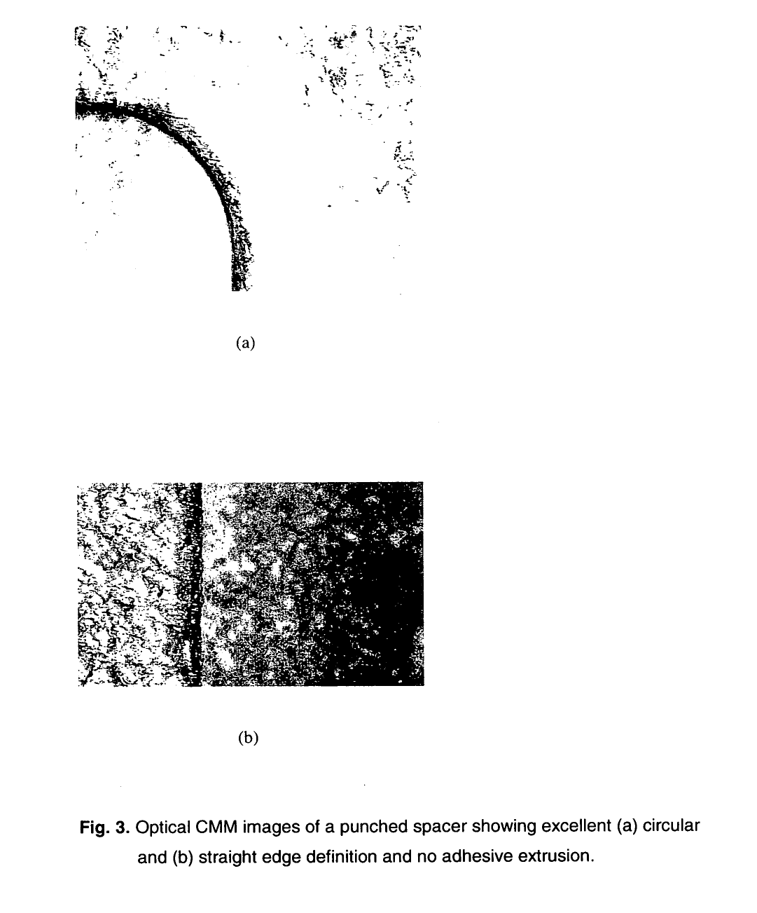

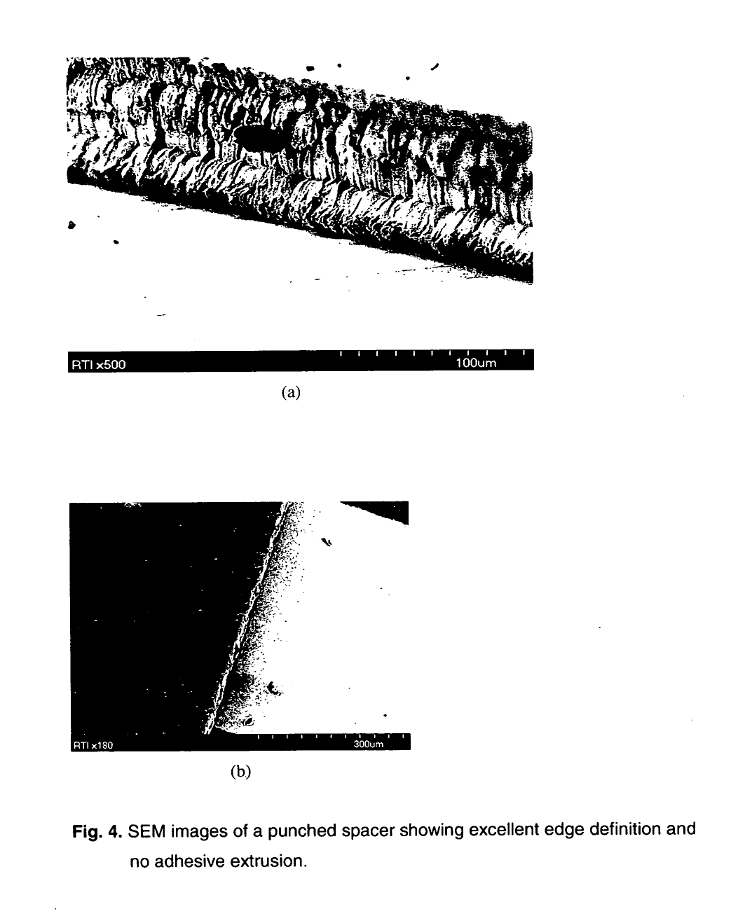

In certain embodiments, the spacer material has at least one hole punched through it, wherein the hole defines a well when placed on the substrate. In various embodiments, the hole may be punched in any configuration or punched multiple times to depending on the desired shape and/or size. For example, as shown in FIGS. 2-4, the punched spacer material according to the present disclosure exhibits excellent edge definition with no adhesive extrusion whether straight or circular patterns are punched through it.

The biosensor also may comprise a reaction reagent system located in the well. Typically in electrochemical sensors the reaction reagent system comprises an electron mediator and an oxidation-reduction enzyme specific for the analyte.

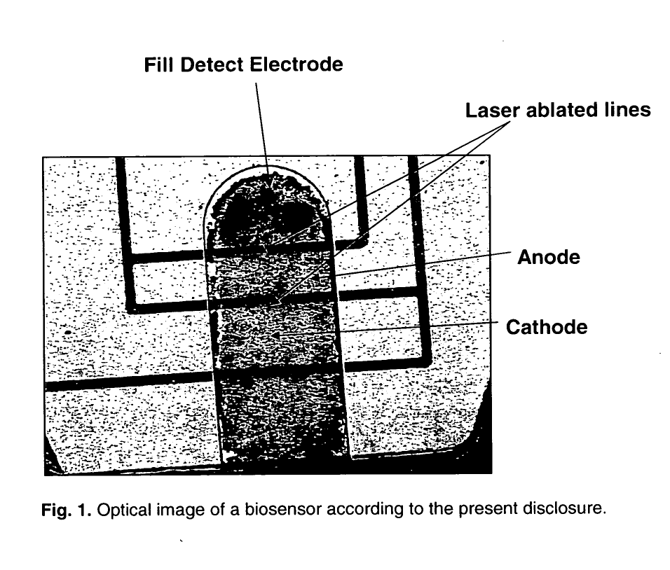

In one embodiment, the heat sealable layer defines two of four edges of the anode. In this embodiment, the two remaining edges of the anode may be defined by lines ablated into the substrate layer by a laser. FIG. 1 shows patterns of lines that are etched into the substrate during sensor fabrication. In this embodiment, the horizontal, parallel lines define two opposing edges of an anode.

One exemplary process is direct writing of electrodes (laser deposition) as described in commonly-assigned, copending provisional patent application No. 60/716,120 “Biosensor with Direct Written Electrode”, filed Sep. 13, 2005, the disclosure of which is hereby incorporated herein by reference in its entirety.

Because of the importance of anode edge definition, the spacer material should meet at least one of the following requirements:
No adhesive extrusion into sample cavity since this would cause variability in anode definition.
Hermetic seal with the electrode material to ensure no leaks of the chemistry solution or blood under the spacer.
No tack prior to activation of the adhesive to avoid the use of a liner that would need to be removed prior to lamination. In addition, the liner could interfere with punched edge quality.
Good punched edge quality, which is a function of the punch tool, punch conditions, and the material. Edge quality is important for anode definition and forming a good seal with the cover material.

In accordance with another aspect of the present disclosure, provided herein are biosensors comprising unique electrode materials, including semiconducting and conducting materials. The conducting materials include traditional metals, as well as novel thin film carbon materials.

When conducting materials are used, the at least one electrode may comprise a metal chosen from or derived from gold, platinum, rhodium, palladium, silver, iridium, carbon, steel, metallorganics, and mixtures thereof. In one embodiment, a carbon electrode can further comprise Cr.

When the at least one electrode is semiconducting, it may comprise a material chosen from tin oxide, indium oxide, titanium dioxide, manganese oxide, iron oxide, and zinc oxide. In one embodiment, the at least one semiconducting electrode comprises zinc oxide doped with indium, tin oxide doped with indium, indium oxide doped with zinc, or indium oxide doped with tin.

In another embodiment, the at least one semiconducting electrode comprises an allotrope of carbon doped with boron, nitrogen, or phosphorous.

As stated, the biosensor disclosed herein includes at least one or more electrodes and a reaction reagent system comprising an electron mediator and an oxidation-reduction enzyme specific for the analyte to be measured. In various embodiments, the analyte may be chosen from glucose, cholesterol, lactate, acetoacetic acid (ketone bodies), theophylline, and hemoglobin A1c.

When the biosensor is used to measure an analyte comprising glucose, the at least one oxidation-reduction enzyme specific for the analyte may be chosen from glucose oxidase, PQQ-dependent glucose dehydrogenase and NAD-dependent glucose dehydrogenase.

In other non-limiting embodiments, the electron mediator may comprise a ferricyamide material, such as potassium ferricyamide, ferrocene carboxylic acid or a ruthenium containing material, such as ruthenium hexaamine (III) trichloride.

The reaction reagent system may also comprise a variety of buffers, surfactants and binders. For example, in one embodiment, the buffer material comprises potassium phosphate. The surfactants may be chosen from non-ionic, anionic, and zwitterionic surfactants. In addition, the polymeric binder may be chosen from hydroxypropyl-methyl cellulose, sodium alginate, microcrystalline cellulose, polyethylene oxide, hydroxyethylcellulose, polypyrrolidone, PEG, and polyvinyl alcohol.

When used to measure analytes in blood, the reaction reagent system typically further comprises a red blood cell binding agent for capturing red blood cells. Such binding agents include lectins.

Depending on the analyte of interest, the reaction reagent system may include such optional ingredients as buffers, surfactants, and film forming polymers. Examples of buffers that can be used in the present invention include without limitation potassium phosphate, citrate, acetate, TRIS, HEPES, MOPS and MES buffers. In addition, typical surfactants include non-ionic surfactant such as Triton X-100® and Surfynol®, anionic surfactant and zwitterionic surfactant. Triton X-100® (an alkyl phenoxy polyethoxy ethanol), and Surfynol® are a family of detergents based on acetylenic diol chemistry. In addition, the reaction reagent system may optionally include wetting agents, such as organosilicone surfactants, including Silwet® (a polyalkyleneoxide modified heptamethyltrisiloxane from GE Silicones).

The reaction reagent system further optionally comprises at least one polymeric binder material. Such materials are generally chosen from the group consisting of hydroxypropyl-methyl cellulose, sodium alginate, microcrystalline cellulose, polyethylene oxide, polyethylene glycol (PEG), polypyrrolidone, hydroxyethylcellulose, or polyvinyl alcohol.

In one embodiment, 0.01 to 0.3%, such as 0.05 to 0.25% of a non-ionic surfactant such as Triton X-100 may be used in combination with 0.1 to 3%, such as 0.5 to 2.0% of a polymeric binder material.

Other optional components include dyes that do not interfere with the glucose reaction, but facilitates inspection of the deposition. In one non-limiting embodiment, a yellow dye (fluorescein) or a blue dye (Cresyl Blue) may be used.

In addition to the enzyme specific for the analyte and the electron mediator, the reaction reagent system mentioned above may also include the previously described optional components, including the buffering materials, the polymeric binders, and the surfactants. The reagent layer generally covers at least part of the working electrode as well as the counter electrode.

In one embodiment, by using a reel-to-reel process, multiple biosensors of the type disclosed herein are formed on a sheet of material that serves as the substrate. The other components in the finished biosensor are then built up layer-by-layer on top of the substrate to form the finished product.

The process for making the disclosed biosensors may begin by depositing an electroactive on a plastic substrate. As used herein, an “electroactive” material is intended to mean electrically conducting or semiconducting material.

For example, the electrically conducting material may comprise a metal chosen from or derived from gold, platinum, rhodium, palladium, silver, iridium, carbon, steel, metallorganics, and mixtures thereof. In one embodiment, a carbon electrode can further comprise Cr.

When the at least one electrode is semiconducting, it may comprise a material chosen from tin oxide, indium oxide, titanium dioxide, manganese oxide, iron oxide, and zinc oxide. In one embodiment, the at least one semiconducting electrode comprises zinc oxide doped with indium, tin oxide doped with indium, indium oxide doped with zinc, or indium oxide doped with tin.

In another embodiment, the at least one semiconducting electrode comprises an allotrope of carbon doped with boron, nitrogen, or phosphorous.

The conducting or semiconducting material may be deposited in a known fashion, such as by sputtering a layer ranging from 10 nm to 100 nm. In one non-limiting embodiment, a thin film of gold ranging from 25 nm to 35 nm is deposited onto the plastic substrate.

Desired patterns are next formed onto the substrate by ablating the conducting or semiconducting layer using a focused laser beam. In one embodiment, mirrors are used to direct the laser beam to ablate the material according to a desired pattern. As shown in FIG. 1, the lines etched or ablated by the laser form at least two opposing sides of the anode. The remaining two sides are formed by the spacer material described herein, and particularly exemplified below.

The spacer material according to the present invention is then applied to substrate. Unlike traditional spacer materials in which the underside was coated with an adhesive to facilitate attachment to the dielectric layer and substrate, the inventive spacer material does not require an adhesive. Rather, a pre-punched spacer material according to the present disclosure bonds to the substrate by a heat sealable layer.

As stated, prior to being applied to the substrate, at least one hole is punched through the spacer material. FIGS. 2-4 show various SEM and optical images of punched spacer material according to the present disclosure. As shown in these figures, the punched spacer material exhibits excellent edge definition with little or no adhesive extrusion. Adhesive extrusion is defined as poor edge definition resulting from adhesion of the spacer material to the punch tool used to form the hole. What is also evident from these figures in the uniformity of the coating on the substrate.

After the punching process, the spacer material is positioned on the substrate such that it covers at least a portion of the anode. In one embodiment, the spacer material defines two edges of the anode. In this embodiment, the two edges that define the anode edges are those that have been punched. In order to accurately define the area of the anode, it is desirable to have excellent edge definition after punching the spacer. In another embodiment, the spacer material may be applied to the substrate such that it also covers a portion of the electrode, or cathode, or a portion of both the electrode and cathode.

After the spacer material is applied to the substrate in the manner described, it is laminated to the substrate to ensure a hermetic seal with the electrode material. If done properly, there will be no leaks of the chemistry solution or blood under the spacer. The laminating procedure is typically performed at a temperature ranging from 250 to 300° F. and pressure ranging from 5 to 60 psi.

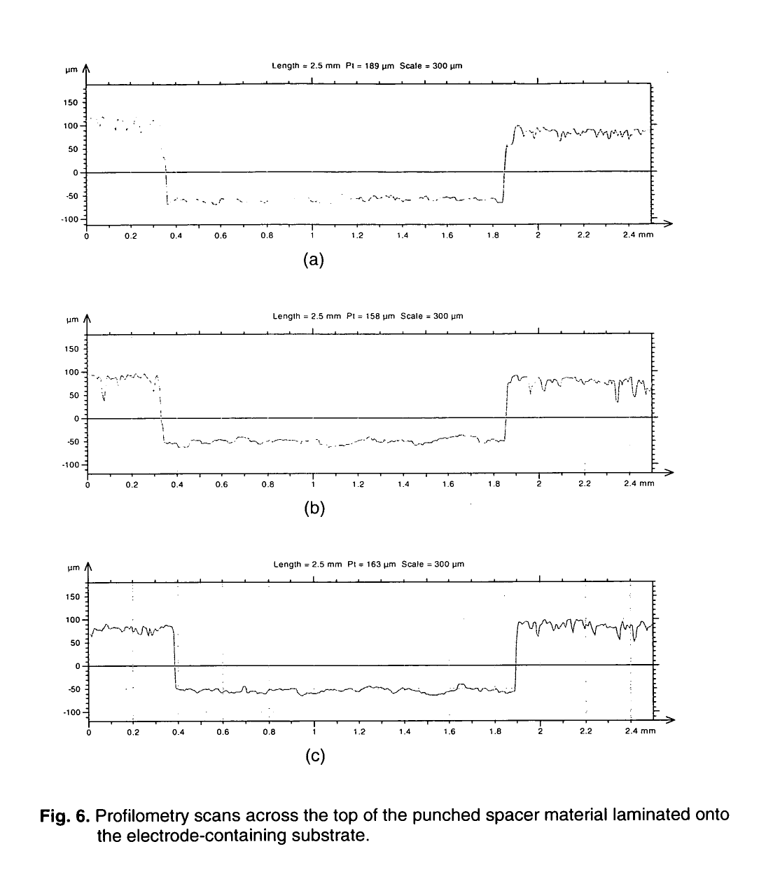

The laminated biosensor shows a uniformly smooth surface with a excellent edge definition for the anode. The uniformity in the coating and anode edge definition is exemplified in the profilometry scans provided in FIG. 6. These scans were taken across the top of the punched spacer material laminated onto the electrode-containing substrate and show a minimal edge slope between the surface and the cavity and absence of burrs or other defects along punched edges.

In one embodiment, after laminating the spacer to the substrate, the assembled sensor comprises an anode, cathode, and two fill detect electrodes, with the anode area defined on two opposing sides by laser ablation of the underlying conducting or semiconducting material, and the two remaining sides by the punched spacer.

In addition, the at least one hole punched through the spacer defines a cavity or well sufficient for receiving certain chemistries after lamination. Chemistry can be deposited into the cavities or wells of the assembled biosensor using a variety of methods, including piezo dispensing, micropipetting, or spray coating.

In one embodiment, a reagent system comprising an electron mediator and an oxidation-reduction enzyme specific for the analyte is applied to the biosensor. An aqueous composition comprising the reagent system can be applied via the previously mentioned techniques, onto exposed portion of the working electrode and drying it to form reagent layer.

The aqueous composition comprising the reagent system can include an electron mediator chosen from a ferricyamide material, ferrocene carboxylic acid or a ruthenium containing material. In one embodiment, the ferricyamide material comprises potassium ferricyamide and the ruthenium containing material comprises ruthenium hexaamine (III) trichloride.

The deposited reaction reagent system further comprises at least one buffer material, such as one comprising potassium phosphate.

The reaction reagent system may also comprise a variety of buffers, surfactants and binders. For example, in one embodiment, the buffer material comprises potassium phosphate. The surfactants may be chosen from non-ionic, anionic, and zwitterionic surfactants. In addition, the polymeric binder may be chosen from hydroxypropyl-methyl cellulose, sodium alginate, microcrystalline cellulose, polyethylene oxide, hydroxyethylcellulose, polypyrrolidone, PEG, and polyvinyl alcohol.

In one non-limiting embodiment, the reaction reagent system comprises 0.01 to 0.3% of a non-ionic surfactant, such as 0.05 to 0.25% of an alkyl phenoxy polyethoxy ethanol, and 0.1 to 3%, of a polymeric binder material, such as 0.5 to 2.0% of polyvinyl alcohol.

A transparent cover may then be attached to top of the spacer to form the sample cavity.

In an embodiment, a secondary redox probe (“SRP”) may be added to the biosensor chemistry. For purposes of this disclosure, “redox probe” means a substance capable being oxidized and/or reduced.

It is possible for the secondary redox probe to comprise an additional electron mediator substance capable of undergoing an electrochemical redox reaction. Accordingly, in the same manner as the ruthenium hexaamine mediator mentioned above, the secondary redox probe substance generates a current in response to the application of a voltage pulse. The secondary redox probe, however, differs from the ruthenium hexaamine (i.e. the primary redox probe), or the other mediators cited above, in that the current generated is unrelated to the glucose concentration, but still dependent on the particular blood level of the sample, particularly the hematocrit level (i.e. the percentage of the amount of blood that is occupied by red blood cells) of the sample.

Accordingly, the electrochemical signal produced by the SRP will be a function of the hematocrit of the sample, but not glucose dependant, and it will therefore function as an internal standard for hematocrit evaluation.

Some of the classes of compounds that could function as a SRP include transition metal complexes, such as ferrocene derivatives, simple ions, such as Fe(III) and Mn(II), organometallics, organic dyes, such as cresyl blue, simple organics, such as such as gentisic acid (2,4-benzoic acid), and trihydrohybenzoic acid, and other organic redox-active molecules, such as peptides containing redox-active amino acids, and particles on the order of nm in size that contain redox-active components.

The following is an exemplary list of characteristics that the SRP may exhibit:
little or no interference with the glucose measurement (i.e., limited interaction with the enzyme, mediator, or glucose);
oxidized or reduced in a potential range that can be easily distinguished from that of the mediator;
soluble in the strip chemistry formulation; and
little or no interference with stability of the sensor, or any other performance parameter.

For an electrochemically active compound to be useful as an SRP, it desirable to have a potential distinctly different from the primary mediator, but not so extreme that measuring it would result in a noisy signal due to interference. For example, when ruthenium hexaamine is used as the mediator, there are generally two ‘windows’ in the potential range. In an oxidation based approach, one of the windows is from about 0.3 to approximately 0.9V. The second window is the reduction-based technique, and extends from approximately −0.15V to −0.5V. It is important to remember that the numbers cited here are only for a very specific example, and should not be construed as a general rule. There may be cases where an SRP that has a peak at 0.2V, or at other magnitudes, would be perfectly acceptable. The actual range of the windows is dependent on the potential required for the primary measurement.

Beyond the scope of hematocrit dependence, potential ranges, and a preference for avoiding interference with the primary measurement, there are few restrictions on what exactly can be used as an SRP. This enables the use of a wide variety of substances, including, but not limited to: simple organics, macromolecules, functionalized microbeads, transition metal complexes, nano-particles, and simple ions.

The present disclosure is further illuminated by the following non-limiting examples, which are intended to be purely exemplary of the invention.

### EXAMPLES

The following examples describe the fabrication and testing of biosensors according to one embodiment of the present disclosure. In these examples, the biosensor had ablated electrodes with punched spacer laminated onto it. Example 1 describes tests performed to determine the precision (geometric and surface roughness) of anode areas on biosensors that do not have any chemistry on them. Example 2 provides blood testing data of biosensors that further comprise chemistry.

### Example 1

A thin film of gold (30 nm) was sputtered onto a plastic film substrate (PET). The gold layer was then laser ablated using a focused beam approach, in which Galvo mirrors were used to direct the laser beam to ablate the material according to a desired electrode pattern. The remaining gold layer was formed into desired patterns for an electrode array, which included an anode, cathode, and two fill detect electrodes.

Next, the second layer or spacer layer of the biosensor was formed by first punching out sample cavities in a polyester film having a heat seal coating. The polyester film used for the spacer was a commercially available PET film (3M Scotchpak™ MA370M), which had a total thickness of 3.7 mils, including the heat seal coating of 0.8 mils.

The punched spacer material was laminated onto laser ablated electrode substrate to form assembled biosensors having an anode, cathode and two fill detect electrodes. As shown in FIG. 1, the anode area was defined on two sides by the laser ablation of the gold layer, and the other two by the sample cavities punched out of the spacer.

In addition to the ablated electrodes and the spacer described above, a chronoamperometry solution comprising 5 mM ferrocyamide and 200 mM ferricyamide in 100 mM phosphate buffer, with 0.1% of Triton X-100 was applied to the samples. The biosensor had no other chemistry or cover.

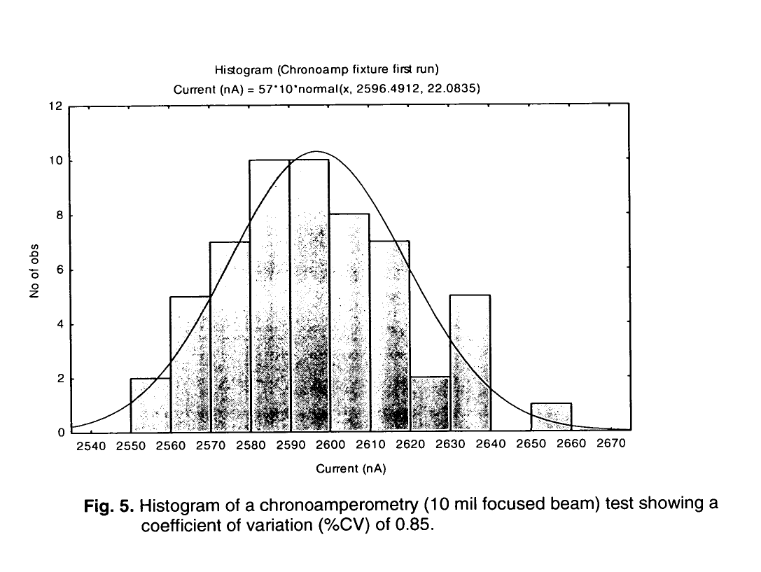

The biosensors fabricated were analyzed using chronoamperometry which allowed reproducibility of the anode area to be determined. As shown in FIG. 5, coefficient of variation (% CV) is 0.85, which was essentially the error of the measurement of the instrument, indicating that all 57 sensors tested according to this example were almost identical. As evident, % CV values, which determines precision in anode area, illustrates excellent reproducibility of both laser ablation and punched spacer definition, the two boundaries that define the anode.

### Example 2

Once the sensors were assembled according to Example 1, chemistry was dispensed into the sample cavities using micropipetting. Blood volume required to fill the sample cavity of this biosensor was 0.25 ul when a 100 μm thick spacer layer was used. Table 1 below shows the relative percentages by weight of the various ingredients dispensed into the sample cavities.

TABLE 1

| Ingredient | Weight Percent |
|---|---|
| Phosphate, Monobasic (Buffer) | 0.64% |
| Phosphate, Dibasic (Buffer) | 0.92% |
| Silwet L-7206 (Spreading Agent) | 0.051% |
| Triton-X 100 (Spreading Agent) | 0.051% |
| Methocel F4M (Binder) | 20.00% |
| Sucrose (Enzyme Stabilizer) | 5.00% |
| Hexammine Ruthenium (III) Chloride (Mediator) | 5.88% |
| PQQ Dependent Glucose Dehydrogenase (Enzyme) | 10.00% |
| 18 mega ohm deionized water | Balance |

The chemistry solution was then dried and a cover was applied over the sample cavities to form capillary gaps into which blood sample could be drawn. Blood testing data was taken on the finished samples, with sample sizes ranging from 40-60 per blood level for the values shown in Table 2. As in Example 1, coefficient of variation (% CV) was both low and uniform across the measured blood levels indicating a high degree of precision for the tested samples.

TABLE 2

| YSI | | | | | | | | | |
|---|---|---|---|---|---|---|---|---|---|
| reading | 30 mg/dl | 62 mg/dl | 84 mg/dl | 109 mg/d | 168 mg/dl | 243 mg/dl | 301 mg/dl | 398 mg/d | 588 mg/dl |
| Average | 1411 | 2365 | 2850 | 3728 | 5383 | 7376 | 8763 | 10884 | 14935 |
| current | | | | | | | | | |
| (nA) | | | | | | | | | |
| StDev | 68 | 79 | 94 | 221 | 235 | 149 | 187 | 356 | 549 |
| % CV | 4.8 | 3.3 | 3.3 | 5.9 | 4.4 | 2.0 | 2.1 | 3.3 | 3.7 |
| Average | 18 | 57 | 77 | 113 | 178 | 260 | 317 | 403 | 565 |
| glucose | | | | | | | | | |
| (mg/dL) | | | | | | | | | |
| StDev | 2.2 | 3.0 | 3.9 | 5.3 | 7.0 | 6.1 | 6.9 | 13.9 | 20.5 |
| % CV | 12.4 | 5.3 | 5.0 | 4.7 | 3.9 | 2.3 | 2.2 | 3.4 | 3.6 |

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A biosensor for measuring analyte in a fluid, said biosensor comprising: a substrate layer, said substrate layer comprising:
at least one electrode;
at least one cathode;
at least one anode;
at least one spacer material, wherein said spacer material comprises a heat sealable organic layer that covers at least a portion of the anode and defines at least one edge of said anode, wherein said spacer material has at least one hole punched through it, said hole defining at least one sample cavity or well;
a reaction reagent system located in said at least cavity or well, said reaction reagent system comprising an electron mediator and an oxidation-reduction enzyme specific for said analyte; and
a cover disposed over the sample cavity or well to form at least one capillary gap into which blood could be drawn.

2. The biosensor of claim 1, wherein said heat sealable organic layer comprises a polyester containing film with a polyolefin layer disposed thereon.

3. The biosensor of claim 2, wherein said polyester containing film comprises polyethylene terephthalate (PET).

4. The biosensor of claim 1, wherein said heat sealable layer activates at or above 85° C.

5. The biosensor of claim 1, wherein said heat sealable layer defines two of four edges of said anode.

6. The biosensor of claim 5, wherein the two remaining edges of the anode are defined by lines ablated into said substrate layer by a laser.

7. The biosensor of claim 1, comprising two or more fill detect electrodes.

8. The biosensor of claim 1, wherein the at least one electrode is conducting and comprises a metal chosen from or derived from gold, platinum, rhodium, palladium, silver, iridium, carbon, steel, metallorganics, and mixtures thereof.

9. The biosensor of claim 8, wherein the at least one carbon electrode further comprising Cr.

10. The biosensor of claim 1, wherein the at least one electrode is semiconducting.

11. The biosensor of claim 10, wherein the semiconducting electrode comprises a material chosen from tin oxide, indium oxide, titanium dioxide, manganese oxide, iron oxide, and zinc oxide.

12. The biosensor of claim 10, wherein the at least one semiconducting electrode comprises zinc oxide doped with indium, tin oxide doped with indium, indium oxide doped with zinc, or indium oxide doped with tin.

13. The biosensor of claim 10, wherein the at least one semiconducting electrode comprises an allotrope of carbon doped with boron, nitrogen, or phosphorous.

14. The biosensor of claim 1, wherein the analyte is chosen from glucose, cholesterol, lactate, acetoacetic acid (ketone bodies), theophylline, and hemoglobin A1c.

15. The biosensor of claim 14, wherein the analyte comprises glucose and the at least one oxidation-reduction enzyme specific for the analyte is chosen from glucose oxidase, PQQ-dependent glucose dehydrogenase and NAD-dependent glucose dehydrogenase.

16. The biosensor of claim 1, wherein the electron mediator comprises a ferricyamide material, ferrocene carboxylic acid or a ruthenium containing material.

17. The biosensor of claim 16, wherein the ferricyamide material comprises potassium ferricyamide and the ruthenium containing material comprises ruthenium hexaamine (III) trichloride.

18. The biosensor of claim 1, wherein the reaction reagent system further comprises at least one buffer material comprising potassium phosphate.

19. The biosensor of claim 1, wherein the reaction reagent system further comprises at least one surfactant chosen from non-ionic, anionic, and zwitterionic surfactants.

20. The biosensor of claim 1, wherein the reaction reagent system further comprises at least one polymeric binder chosen from hydroxypropyl-methyl cellulose, sodium alginate, microcrystalline cellulose, polyethylene oxide, hydroxyethylcellulose, polypyrrolidone, PEG, and polyvinyl alcohol.

21. The biosensor of claim 1, wherein the reaction reagent system comprises 0.01 to 0.3% of a non-ionic surfactant and 0.1 to 3%, of a polymeric binder material.

22. The biosensor of claim 1, wherein the reaction reagent system comprises 0.05 to 0.25% of an alkyl phenoxy polyethoxy ethanol and 0.5 to 2.0% of polyvinyl alcohol.

23. The biosensor of claim 1, wherein the reaction reagent system comprises one or more secondary redox probes chosen from transition metal complexes, simple ions, organometallics, organic dyes, simple organics, and organic redox-active molecules.

24. The biosensor of claim 23, wherein the transition metal complexes comprise ferrocene derivatives, the simple ions comprise Fe(III) or Mn(II), the organic dyes comprise cresyl blue, the simple organics comprise gentisic acid (2,4-benzoic acid), and trihydrohybenzoic acid, and the organic redox-active molecules comprise peptides containing redox-active amino acids, and particles on the order of nm in size that contain redox-active components.

25. The biosensor of claim 1, wherein the heat sealable organic layer covers at least a portion of the electrode, or cathode, or a portion of both the electrode and cathode.

26. A method of making a biosensor for measuring an analyte, said method comprising:
applying an electroactive material onto a substrate to form a coated substrate;
forming patterns into said coated substrate layer by ablating the electroactive material with a laser, wherein said patterns form an electrode array comprising at least one electrode, cathode, and anode;
applying an organic film on said substrate such that it covers at least a portion of said patterns, wherein at least one hole has been punched into said organic film prior to depositing it onto said substrate, said hole forming at least one well when deposited onto said substrate, wherein said organic film comprises a heat sealable layer that covers at least a portion of the anode and defines at least one edge of said anode;
laminating said organic film onto said substrate by applying heat and pressure to said organic film; and
depositing within said at least one well a reaction reagent system comprising an electron mediator and an oxidation-reduction enzyme specific for said analyte; and
optionally applying a cover to form a capillary for sample application.

27. The method of claim 26, wherein said electroactive material is deposited by sputtering.

28. The method of claim 27, wherein said electroactive material comprises a conducting or semiconducting material.

29. The method of claim 28, wherein said conducting material comprises a metal chosen from or derived from gold, platinum, rhodium, palladium, silver, iridium, carbon, steel, metallorganics, and mixtures thereof.

30. The method of claim 29, wherein the at least one carbon electrode further comprising Cr.

31. The method of claim 28, wherein the semiconducting material is chosen from tin oxide, indium oxide, titanium dioxide, manganese oxide, iron oxide, and zinc oxide.

32. The method of claim 31, wherein the semiconducting material comprises zinc oxide doped with indium, tin oxide doped with indium, indium oxide doped with zinc, or indium oxide doped with tin.

33. The method of claim 28, wherein the semiconducting material comprises an allotrope of carbon doped with boron, nitrogen, or phosphorous.

34. The method of claim 26, wherein the electron mediator comprises a ferricyamide material, ferrocene carboxylic acid or a ruthenium containing material.

35. The method of claim 34, wherein the ferricyamide material comprises potassium ferricyamide and the ruthenium containing material comprises ruthenium hexaamine (III) trichloride.

36. The method of claim 26, wherein the reaction reagent system further comprises at least one buffer material comprising potassium phosphate.

37. The method of claim 26, wherein the reaction reagent system further comprises at least one surfactant chosen from non-ionic, anionic, and zwitterionic surfactants.

38. The method of claim 26, wherein the reaction reagent system further comprises at least one polymeric binder chosen from hydroxypropyl-methyl cellulose, sodium alginate, microcrystalline cellulose, polyethylene oxide, hydroxyethylcellulose, polypyrrolidone, PEG, and polyvinyl alcohol.

39. The method of claim 26, wherein the reaction reagent system comprises 0.01 to 0.3% of a non-ionic surfactant and 0.1 to 3%, of a polymeric binder material.

40. The method of claim 26, wherein the reaction reagent system comprises 0.05 to 0.25% of an alkyl phenoxy polyethoxy ethanol and 0.5 to 2.0% of polyvinyl alcohol.

41. The method of claim 26, wherein the reaction reagent system comprises one or more secondary redox probes chosen from transition metal complexes, simple ions, organometallics, organic dyes, simple organics, and organic redox-active molecules, and combinations thereof.

42. The method of claim 41, wherein the transition metal complexes comprise ferrocene derivatives, the simple ions comprise Fe(III) or Mn(II), the organic dyes comprise cresyl blue, the simple organics comprise gentisic acid (2,4-benzoic acid), and trihydrohybenzoic acid, and the organic redox-active molecules comprise peptides containing redox-active amino acids, and particles on the order of nm in size that contain redox-active components.

43. The method of claim 26, wherein said laminating of the organic film onto said substrate is performed at a temperature ranging from 300 to 400° F. and pressure ranging from 20 to 60 psi.

44. A biosensor for measuring glucose levels in blood, said biosensor comprising:
a substrate layer, said substrate layer comprising:
at least one electrode;
at least one cathode;
at least one anode;
at least one spacer material that comprises a polyethylene terephthalate (PET) with a polyolefin layer disposed thereon, wherein said spacer material activates at or above 85° C., and defines two of four edges of said anode, the two remaining edges of the anode being defined by lines ablated into said substrate layer by a laser,
wherein said spacer material has at least one hole punched through it, said hole defining a sample cavity or well;
a reaction reagent system located in said cavity or well, said reaction reagent system comprising an electron mediator chosen from a ferricyamide material, ferrocene carboxylic acid or a ruthenium containing material, and an oxidation-reduction enzyme chosen from glucose oxidase, PQQ-dependent glucose dehydrogenase and NAD-dependent glucose dehydrogenase; and
a cover disposed over the sample cavity or well to form at least one capillary gap into which blood could be drawn.

45. The biosensor of claim 44, wherein the reaction reagent system comprises one or more secondary redox probes chosen from transition metal complexes, simple ions, organometallics, organic dyes, simple organics, and organic redox-active molecules.

46. The biosensor of claim 45, wherein the transition metal complexes comprise ferrocene derivatives, the simple ions comprise Fe(III) or Mn(II), the organic dyes comprise cresyl blue, the simple organics comprise gentisic acid (2,4-benzoic acid), and trihydrohybenzoic acid, and the organic redox-active molecules comprise peptides containing redox-active amino acids, and particles on the order of nm in size that contain redox-active components.
